(19) United States
(12) Patent Application Publication
(10) Pub. No.: US 2011/0151017 A1
(43) Pub. Date: Jun. 23, 2011

(54) OPHTHALMIC SOLUTIONS WITH IMPROVED DISINFECTION PROFILES

(75) Inventors: Howard Allen Ketelson, Dallas, TX (US); Roya Nicole Borazjani, Dallas, TX (US); Nissanke L. Dassanayake, Fort Worth, TX (US); James W. Davis, Argyle, TX (US); Thomas Christopher Carey, Fort Worth, TX (US); John C. Baker, Fort Worth, TX (US)

(73) Assignee: ALCON RESEARCH, LTD., Fort Worth, TX (US)

(21) Appl. No.: 12/972,095

(22) Filed: Dec. 17, 2010

Related U.S. Application Data
(60) Provisional application No. 61/287,231, filed on Dec. 17, 2009.

(51) Int. Cl. A61K 33/40; A61P 27/02

(52) U.S. Cl. 424/616

(57) ABSTRACT

The present invention relates to ophthalmic solutions with antimicrobial activity. The solutions have an antimicrobial compound such as hydrogen peroxide and a boron compound. In one embodiment, the solutions contain a boron compound such as sodium borate that provides antimicrobial activity in addition to that of hydrogen peroxide, particularly during periods following disinfection and neutralization of such solutions. This additional activity reduces the likelihood of microbial growth in contact lens disinfection applications that neutralize hydrogen peroxide.

### CROSS-REFERENCE TO RELATED APPLICATION

This application claims priority under 35 U.S.C. §119 to U.S. Provisional Patent Application No. 61/287,231, filed Dec. 17, 2009, the entire contents of which are incorporated herein by reference.

### TECHNICAL FIELD OF THE INVENTION

The present invention relates to methods for improving the antimicrobial properties of ophthalmic compositions. The present invention further relates to ophthalmic compositions comprising hydrogen peroxide and a boron compound.

### BACKGROUND OF THE INVENTION

The disinfection of ophthalmic products such as contact lenses often employs compositions comprising antimicrobial agents that are incompatible with ocular tissue when released into the eye during wear. Accordingly, many such compositions utilize antimicrobial agents at low concentrations to avoid toxicity, despite the risk that such concentrations will allow the survival or growth of undesired organisms. In some approaches, multiple agents can be combined to provide an acceptable aggregate level of antimicrobial activity.

Another approach for contact lens disinfection is to utilize a process for disinfection where a composition with a high concentration of an antimicrobial is “neutralized” over a period of time by degrading or otherwise reducing the concentration of the antimicrobial. In this manner, a neutralized composition is formed with an ocular tissue-compatible concentration of antimicrobials. For example, U.S. Pat. No. 3,912,451 describes the neutralization of a phosphate-buffered hydrogen peroxide solution using a transition metal catalyst such as platinum. Unfortunately, the post-neutralized solution can provide the opportunity for any surviving microbes to replicate and grow in the neutralized solutions. Many current peroxide disinfecting solutions contain phosphate buffer systems and/or cellulosic polymer tablet systems, and/or enzymes or other proteins that can serve as nutrient sources for microbial growth. Contact lenses left in these neutralized solution for extended periods may be exposed to unacceptable levels of contaminants, and could serve as vectors to transfer pathogenic microbes to the corneal surface once the lenses are instilled onto the eye, particularly if sterility is compromised through improper handling of contact lenses or lens cases. Accordingly, methods are needed to preserve and reduce the likelihood of antimicrobial growth in solutions having low concentrations of antimicrobials such as, for example, post-neutralized hydrogen peroxide solutions.

Boron compounds such as borates are common excipients in ophthalmic compositions due to good buffering capacity at physiological pH and well known safety and compatibility with a wide range of drugs and preservatives. Borates also have inherent bacteriostatic and fungistatic properties, and therefore aid in the preservation of the compositions.

U.S. Patent Pub. No. 2005/0244509 (Tsao et al.) describes the use of hydrogen peroxide at low concentrations (0.001% to 0.01% by weight) by itself or in combination with other antimicrobials in ophthalmic disinfectants that do not require neutralization. The use of borate as a tonicity or buffering agent is also disclosed.

U.S. Patent Pub. No. 2007/0104798 (Karagoezian) describes the use of low concentrations of peroxy compounds such as hydrogen peroxide (0.005% to 0.05% by weight) in combination with a chlorite compound and relatively low concentrations of boric acid (0.15% to 0.3% by weight).

The prior art generally teaches the use of borates as tonicity or buffering agents in combination with low concentrations of hydrogen peroxide. However, the prior art does not disclose the use of boron compounds to reduce the likelihood of microbial growth in post-neutralized hydrogen peroxide compositions, and particularly not in hydrogen peroxide compositions having low ionic strength and pH.

### BRIEF SUMMARY OF THE INVENTION

The present invention relates to ophthalmic compositions comprising hydrogen peroxide and a boron compound. The compositions of the present invention have antimicrobial activity against ophthalmic pathogens such as C. parapsilosis and S. aureus.

The present inventors have unexpectedly discovered that ophthalmic compositions at neutral pH and ionic strength comprising hydrogen peroxide and a boron compound have desirable disinfection profiles. The incorporation of boron compounds into ophthalmic compositions comprising hydrogen peroxide can prevent microbial growth once the hydrogen peroxide is neutralized, degraded, or otherwise decreases in concentration or effectiveness over time. Incorporating boron into ophthalmic solutions of hydrogen peroxide also offers other advantages.

For instance, in one embodiment of the present invention, a boron buffering system consisting of sodium borate and boric acid is used in a hydrogen peroxide solution at neutral pH to impart post-neutralization antimicrobial properties. In a composition at neutral pH, a boron buffer system would not be expected to impart significant antimicrobial activity due to the high pKa (9.14 at 25° C.) of the system. Unexpectedly, the inventors have found that peroxide solutions buffered with such a boron buffering system have desirable antimicrobial properties even at neutral pH.

Without being bound to theory, it is believed that boron compounds such as boric acid and borates combine with peroxide to form perborate species which act as antimicrobial and cleaning agents. Perborates are rapidly formed in solutions of hydrogen peroxide and boron compounds, even at neutral pH. In aqueous solutions such as the embodiment described above, borate exists in many forms, and in acid and neutral pH conditions, it is boric acid (H₃BO₃ but more correctly B(OH)₃). Boric acid does not dissociate in aqueous solution, but is acidic due to its interaction with water molecules, forming tetrahydroxyborate:

B(OH)₃+H₂O→B(OH)₄+H⁺

Kₐ=5.8×10⁻¹⁰ mol/l; pKₐ=9.24.

Borates can produce peroxoanions by reaction with anions; for example, the reaction between borax and hydrogen peroxide leads to sodium perborate:

Na₂B₄O₇+4H₂O₂+2NaOH→2Na₂B₂O₄(OH)₄+H₂O.

Preferred embodiments of the present invention are ophthalmic compositions comprising hydrogen peroxide and a boron compound such as boric acid and/or sodium borate. In such compositions, the boron compound is present at a concentration of 0.05M to 0.15M and the composition has a pH of 6.5 to 9.0, and more preferably a pH of 7.0 to 7.9, and most preferably 7.0 to 7.5.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings and wherein:

FIG. 1 is a graph presenting the results of a post-neutralization latency assay for C. parapsilosis comparing several ophthalmic compositions for contact lens disinfection and test solutions;

FIG. 2 is a graph presenting the results of a post-neutralization latency assay for E. coli comparing several ophthalmic compositions for contact lens disinfection and test solutions;

FIG. 3 is a graph presenting the results of a post-neutralization latency assay for S. aureus comparing several ophthalmic compositions for contact lens disinfection and test solutions;

FIG. 4 is a graph presenting the results of a post-neutralization latency assay for C. parapsilosis comparing several ophthalmic compositions for contact lens disinfection and a test solution;

FIG. 5 is a graph presenting the results of a post-neutralization latency assay for E. coli comparing several, ophthalmic compositions for contact lens disinfection and a test solution; and

FIG. 6 is a graph presenting the results of a post-neutralization latency assay for S. aureus comparing several ophthalmic compositions for contact lens disinfection and a test solution.

### DETAILED DESCRIPTION OF THE INVENTION

The ophthalmic compositions of the present invention comprise hydrogen peroxide and a boron compound. The boron compounds which may be used in the compositions of the present invention are boric acid and other pharmaceutically acceptable alkali metal, alkaline earth metal, and transition metal salts such as sodium borate (borax) and potassium borate. As used herein, the term “boron compound” refers to all pharmaceutically suitable compounds comprising boron. As used herein, the term “boron compound” shall include, without limitation, boric acid, salts of boric acid, other pharmaceutically acceptable borates, boric acid, sodium borate, potassium borate, calcium borate, magnesium borate, manganese borate, and other such borate salts.

The amount of hydrogen peroxide contained, in the ophthalmic compositions will vary, as described above, but will generally be in the amount of from 0.1 to 3.5% (w/v); preferred concentrations are from 2.5 to 3.5% (w/v). The total boron concentration (mols of elemental boron per liter) of the compositions of the present invention is generally between 0.05M to 0.15M. In preferred embodiments, the total boron compound concentration is 0.10M to 0.15M.

The compositions of the present invention optionally comprise one or more excipients. Excipients commonly used in ophthalmic compositions include, but are not limited to, tonicity agents, preservatives, chelating agents, buffering agents, surfactants, antioxidants, solubilizing agents, stabilizing agents (e.g., phosphonic acid and organophosphates such as DEQUEST®), antifoaming agents, stabilizing agents, comfort-enhancing agents, polymers, emollients, pH-adjusting agents, additional disinfecting agents, and/or lubricants. In certain embodiments, excipients are selected on the basis of their inertness towards hydrogen peroxide.

Suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants, antifoaming agents, comfort-enhancing agents and polymers include, but are not limited to, ionic and nonionic surfactants, though nonionic surfactants are preferred, hydroxypropyl methylcellulose, guar and polyoxyethylene-polyoxybutylene (PEO-PBO) copolymers. Certain embodiments of the present invention comprise PEO-PBO copolymers such as those described in co-pending U.S. patent application Ser. No. 11/953,654 (U.S. Patent Pub. No. 2008/0138310) entitled “Use of PEO-PBO Block Copolymers in Ophthalmic Compositions”, the entire contents of which are hereby incorporated by reference. PEO-PBO copolymers used in such embodiments include, but are not limited to, diblock and triblock copolymers (e.g., PEO-PBO-PEO and reverse triblocks such as PBO-PEO-PBO copolymers). The copolymers are generally used in embodiments of the present invention at a concentration of 0.001 to 1.0 w/v %, and preferably at a concentration of 0.001 to 0.1 w/v %.

Certain embodiments of the present invention are ophthalmic compositions comprising hydrogen peroxide and a boron compound that are substantially free of surfactants. These substantially surfactant-free embodiments demonstrate advantageous and unexpected behavior relative to neutralization kinetics, as shown by the data presented below in EXAMPLE 4 below. Surfactant-free peroxide formulations of the present invention may neutralize at a slower rate than those formulations containing surfactants and accordingly retain a higher concentration of hydrogen peroxide during the neutralization process and the attendant antimicrobial advantages. Also, surfactant-free embodiments may also demonstrate unexpected and advantageous cleaning properties, as shown by the lysozyme cleaning data presented in EXAMPLE 5 below.

The ophthalmic compositions of the present invention may comprise one or more additional preservatives, disinfecting, or antimicrobial agents. Examples of such preservatives and agents include, but are not limited to, benzalkonium chloride, sodium perborate, sodium chlorite, guanidine derivatives such as polyhexamethylene biguanide, and quaternary ammonium salts. In certain embodiments, the composition may be self-preserved that no preservation agent is required.

The compositions of the present invention are preferably isotonic, or slightly hypotonic. This may require a tonicity agent to bring the osmolality of the compositions to a level at or near 210-320 milliosmoles per kilogram (mOsm/kg). The compositions of the present invention generally have an osmolality in the range of 210-320 mOsm/kg, and preferably have an osmolality in the range of 220-300 mOsm/kg. The ophthalmic compositions will generally be formulated as sterile aqueous solutions.

Certain compositions described herein may be used to disinfect and/or clean contact lenses in accordance with processes known to those skilled in the art. More specifically, contact lenses are removed from a patient's eyes and then placed in contact with such compositions for a time sufficient to disinfect the lenses. Disinfection and/or cleaning typically requires soaking the lenses in the composition for approximately 4 to 6 hours, during which time neutralization takes place. Neutralization of the hydrogen peroxide in compositions of the present invention can occur using methods known to the art (such as, for example, catalytic or enzymatic methods). Platinum- or catalase-based neutralization methods are preferred for use with the compositions of the present invention. Although not necessary, the solution containing a contact lens can be agitated, for example, by shaking the container containing the composition and contact lens to at least facilitate removal of deposit material from the lens. A contact lens optionally may be manually rubbed with saline or a substantially isotonic solution to remove further deposit material from the lens. The cleaning and disinfecting can also include rinsing the lens prior to returning the lens to a wearer's eye. Embodiments of the invention are usable with many types of contact lenses including, but not limited to, hydrogel soft lenses, silicon hydrogel (SiH) lenses, HEMA lenses, high water content hydrogel HEMA lenses, and rigid gas permeable (RGP) lenses.

Compositions of the present invention may also comprise one or more indicator compounds. Such indicator compounds provide a visual indication when the hydrogen peroxide concentration of the composition has dropped following neutralization to a level acceptable to prevent ocular irritation or discomfort if the composition is instilled into an eye. Many of these indicator compounds are known to the art and include, for example, phenolphthalein or iodine-chromophores such as those disclosed in U.S. Pat. No. 5,603,897 to Heller et al. Compositions of the present invention can also be used with tablet neutralization systems (particularly catalase tablets having indicator systems such as those disclosed in U.S. Pat. No. 6,440,411 to Scherer et al., herein incorporated by reference in its entirety).

The following examples are presented to further illustrate selected embodiments of the present invention.

### Example 1

| Ingredient | % w/v |
|---|---|
| Hydrogen Peroxide | 3.0 |
| Sodium Borate | 0.33 |
| Boric Acid | 0.41 |
| Sodium Phosphate, monobasic | 0.136 |
| monohydrate | |
| Sodium Phosphate, dibasic anhydrous | 0.062 |
| Dequest 2060S | 0.12 |
| Sodium Chloride | 0.47 |
| Sodium Hydroxide and/or hydrochloric | q.s. to adjust to pH 7.0 |
| acid | |
| Purified Water | q.s. 100% |

### Example 2

| Ingredient | % w/v |
|---|---|
| Hydrogen Peroxide | 3.0 |
| Sodium Borate | 0.55 |
| Boric Acid | 0.41 |
| Sodium Phosphate, monobasic | 0.136 |
| monohydrate | |
| Sodium Phosphate, dibasic anhydrous | 0.062 |
| Dequest 2060S | 0.12 |
| Sodium Chloride | 0.47 |
| Purified Water | q.s. 100% |

### Example 3

Compositions of the present invention were tested in a latency assay to compare the differences between boron-containing solutions and neutralized marketed hydrogen peroxide disinfecting solutions. Boron-containing solutions at pH 7 and 7.9 were tested against the marketed OXYSEPT® and CLEARCARE® brand hydrogen peroxide disinfecting solutions, the disinfectant solution UNISOL® 4, and saline (positive control). UNISOL® 4 was used as a negative control, and contains boron at at pH 7.4. The compositions of the four boron-containing test solutions and UNISOL® 4 are detailed in TABLE 1 below.

TABLE 1

| Composition | | | | | | |
|---|---|---|---|---|---|---|
| Chemical | 15101- | 15101- | 15101- | 15101- | 15283- | |
| (% wt/% vol) | 40A | 40B | 40C | 40D | 027 | UNISOL ®4 |
| Hydrogen | 3.0 | 3.0 | — | — | 3.0 | — |
| Peroxide (50% | | | | | | |
| Arkema) | | | | | | |
| Hydrogen | 2.9 ± 0.1 | 2.9 ± 0.1 | — | — | 3.0 ± 0.1 | — |
| Peroxide | | | | | | |
| (Assay) | | | | | | |
| Sodium Borate | 0.33 | 0.55 | 0.33 | 0.55 | 0.33 | 0.052 |
| Boric Acid | 0.41 | 0.27 | 0.41 | 0.27 | 0.41 | 0.5 |
| Sodium | 0.136 | 0.136 | 0.136 | 0.136 | 0.136 | — |
| Phosphate, | | | | | | |
| monobasic | | | | | | |
| monohydrate | | | | | | |
| Dibasic, | 0.062 | 0.062 | 0.062 | 0.062 | 0.062 | — |
| sodium | | | | | | |
| phosphate | | | | | | |
| anhydrous | | | | | | |
| Dequest 2060S | 0.12 | 0.12 | 0.12 | 0.12 | 0.12 | — |
| Sodium | 0.47 | 0.47 | 0.47 | 0.47 | 0.47 | 0.66 |
| Chloride | | | | | | |
| Purified Water | QS | QS | QS | QS | QS | QS |
| | | | | | 100% | |
| pH after | 7.0 | 7.9 | 7.0 | 7.9 | 7.0 | 7.4 |
| neutralization | | | | | | |
| Osmolality | 300 | 300 | 300 | 300 | 300 | 292 |
| after | | | | | | |
| neutralization | | | | | | |
| Total Boron | 0.101M | 0.101M | 0.101M | 0.101M | 0.101M | 0.086M |
| Concentration | | | | | | |
| (mol/L) | | | | | | |

Hydrogen peroxide in samples was assayed according to the following procedure.
1. Pipette 0.1 ml (100 μl) of analytical sample into a 10 ml glass beaker,
2. Add to 5 mL of demineralized water, 2 mL of diluted hydrochloric acid solution, 2 mLs of potassium iodide solution, and 1 ml drops ammonium molybdate solution.
3. Keep sample covered in the dark for ˜5 mins before titrating.
4. Titrate with 0.1 N sodium thiosuffate to faint yellow or straw color. Swirl or stir gently during titration to minimize iodine loss.
5. Add about 1-2 mL starch indicator, and continue titration until the blue color just disappears.
6. Repeat steps 2-4 on a blank sample of water (omitting the H2O2).

The percentage hydrogen peroxide in each sample is calculated using the following formula:

% H₂O₂=(mLs N—Na₂S₂O₃)×(N)×(0.01701)×(ml of sample)×100÷ml of sample
where N=Normality of the standardized potassium iodide.

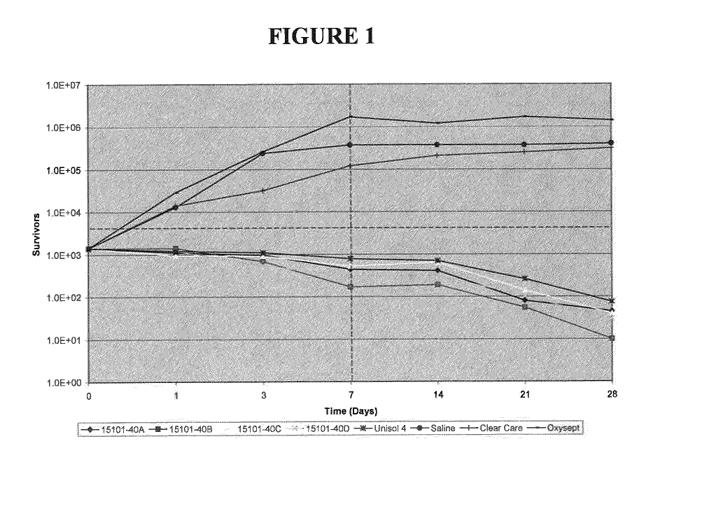

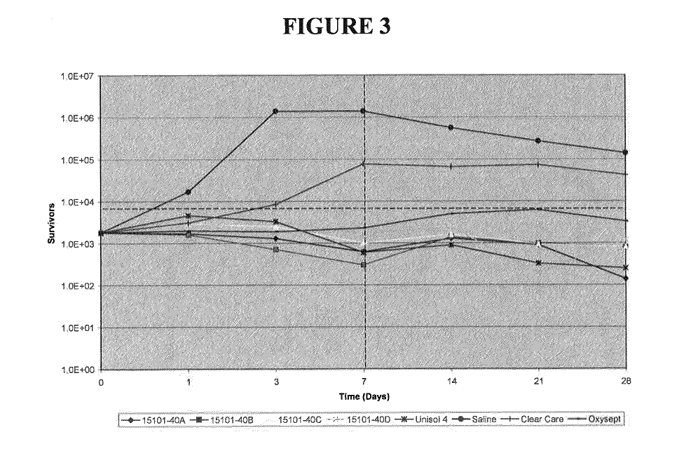

The samples were assayed for antimicrobial activity as follows. Samples of hydrogen peroxide disinfectant solutions are neutralized fully according to label instructions. Following neutralization, a representative contact lens coated with an FDA organic soil is added to the remaining neutralized solution, followed by inoculating with a single strain of microorganism. The selected challenge microorganisms include E. coli (ATCC 48739), S. aureus (ATCC #6538) and C. parapsilosis (ATCC #22019). The neutralized solutions are sampled for the growth of survivors on days 1 through 7. Following the day 7 sample, the neutralized solutions are rechallenged, following with additional sampling at days 14 through 28. The survivors are enumerated over time using a suitable recovery system. The neutralized solution's latency effect is considered adequate if stasis is obtained (no growth occurs, ±0.5 for fungi), indicated by the horizontal dashed line in FIGS. 1-3 presenting the results of the assay.

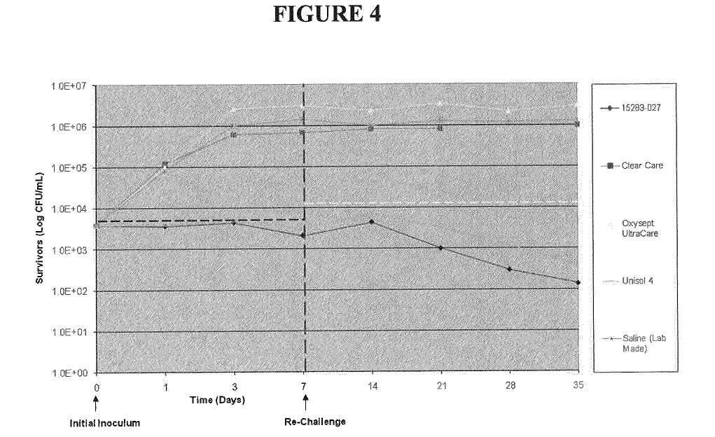

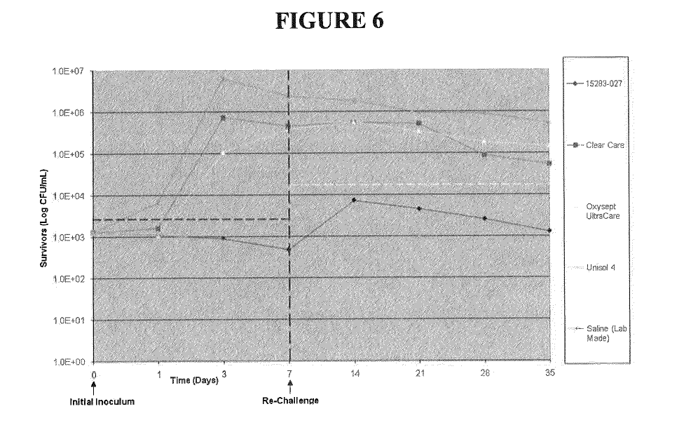

In an alternative test method, selected challenge microorganisms are mixed with the FDA organic soil (100% vol/vol) and two lenses/type were coated with this mixture (50 ul/lens). After 5-10 minutes coated lenses are placed into 10 ml neutralized solution. The neutralized solutions are sampled for the growth of survivors on days 1 through 7. Following the day 7 sample, the neutralized solutions are rechallenged, following with additional sampling at days 14 through 35. The survivors are enumerated over time using a suitable recovery system. The neutralized solution's latency effect is considered adequate if stasis is obtained (no growth occurs, ±0.5 for fungi), indicated by the horizontal dashed line in FIGS. 4-6 presenting the results of the assay.

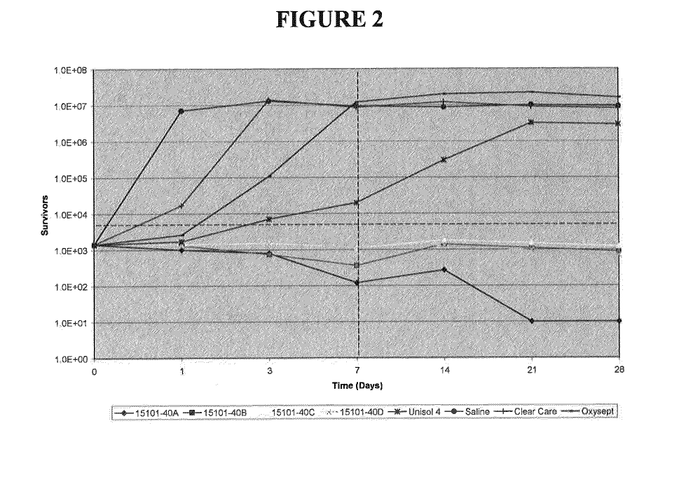

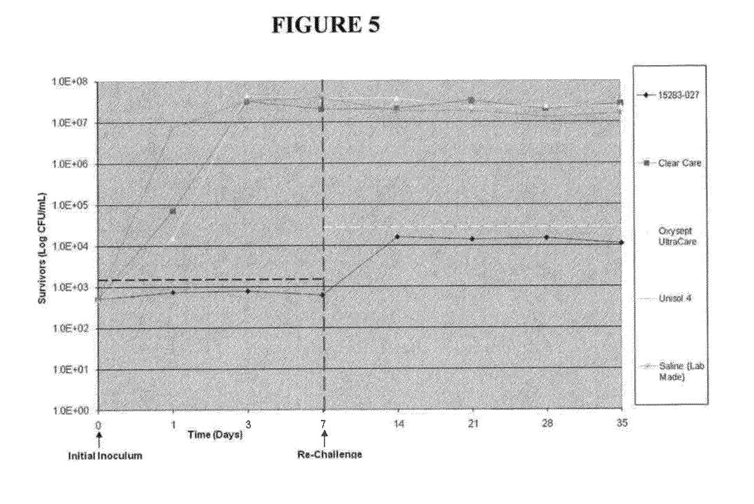

In both studies, neutralized marketed products (OXYSEPT® and CLEARCARE® brand hydrogen peroxide disinfecting solutions) and saline (positive control) supported growth of the tested organisms for up to 35 days. For the C. parapsilosis and E. coli tests, this growth was quite rapid for saline control and neutralized marketed products. The boron only UNISOL® 4 did not allow organism growth for the C. parapsilosis and S. aureus tests (FIGS. 1, 3, 4, and 6, respectively). However, the boron only UNISOL® 4 did allow the gradual growth of E. coli as shown in FIGS. 2 and 5. The hydrogen peroxide and boron systems completely inhibited microbial growth post-neutralization at the pH range tested (7.0-7.9) for all tested organisms. Thus, it appears that the hydrogen peroxide and boron in the compositions of the present invention demonstrate an unexpected post-neutralization antimicrobial profile possibly due to the formation of perborate moieties.

### Example 4

Two 3% hydrogen peroxide formulations were compared in a kinetics assay to evaluate the possible effects of surfactants on platinum-based neutralization of hydrogen peroxide disinfectant solutions. A surfactant-free test hydrogen peroxide solution similar to the composition of EXAMPLE 1 above was compared to CLEARCARE® hydrogen peroxide disinfecting solution, which contains a block copolymer surfactant (PLURONIC® 17R4). In the kinetics assay procedure, 10 mL of test formulation was pipette into a contact lens case. A cap with one of two platinum discs was placed into the case and tightened. At various time points (30, 60, 120, 360, and 1080 minutes) the cap was removed and the solution assayed for hydrogen peroxide. Each solution was neutralized using two different platinum catalysts. The results of the kinetics assay are presented in TABLE 2 below.

TABLE 2
% HYDROGEN PEROXIDE

| Time, | Pt disc 1 using surfactant-free | Pt disc 2 using surfactant-free |
|---|---|---|
| min | peroxide formula | peroxide formula |
| 30 | 0.15 | 0.33 |
| 60 | 0.034 | 0.100 |
| 120 | 0.0180 | 0.0324 |
| 360 | 0.0021 | 0.0035 |
| 1080 | 0.00053 | 0.00074 |
| Time, | | |
| min | Pt disc 1 using CLEARCARE ® | Pt disc 2 using CLEARCARE ® |
| 30 | 0.17 | 0.18 |
| 60 | 0.034 | 0.028 |
| 120 | 0.0085 | 0.0073 |
| 360 | 0.0013 | 0.0014 |
| 1080 | 0.00027 | 0.00056 |

As shown in TABLE 2, the surfactant-free peroxide formulation retained significantly higher concentrations of hydrogen peroxide at all time points with platinum disk 2 compared to the CLEARCARE® formulation with surfactant. The surfactant-free peroxide formulation also retained significantly higher concentrations of hydrogen peroxide at 120, 360, and 1080 minute time points compared to the CLEARCARE® formulation when neutralized with platinum disk 1, and had equivalent concentrations at the 30 and 60 minute time points.

### Example 5

The cleaning properties of a test hydrogen peroxide contact lens disinfecting system similar to the EXAMPLE 1 formulation was evaluated together with two commercial formulations, one containing a surfactant (CLEARCARE®) and the other surfactant free (OXYSEPT®). Lysozyme cleaning efficacy of the test formulation and the two commercial formulations controls was assessed on Acuvue® 2 lenses.

Acuvue® 2 lenses were placed in an 8 mL Wheaton glass sample vial containing 3-mL 1.5 mg/mL Lysozyme solution. The vial is closed with a plastic snap cap and incubated in a constant temperature water bath at 37° C. for 24 hours. After incubation, the soiled lenses are removed from their vials and rinsed by dipping into distilled water. Each soiled lens is placed in the lens basket (2/basket, 2 baskets per solution) in 10 mL of the test solutions at room temperature for 16 hours. After the soaking/cleaning period, the lenses are removed from their respective test solutions and rinsed. The cleaned lenses are then subjected to an extraction procedure in scintillation vials using a trifluoroacetic acid/acetonitrile solution, and quantitative determination of the lysozyme content of the lens extract is carried out by a fluorescence spectrophotometer. The cleaning efficacy of each test solution is calculated by subtracting the amount of lysozyme remaining on each lens from the total amount deposited (as determined by the controls lenses) and then dividing by the total amount multiplied by 100%.

The lysozyme cleaning efficacy of the surfactant-free test solution was 18.0±6.2% which was statistically lower than that of CLEARCARE® (32.7±5.0%), but statistically greater than that of OXYSEPT® (10.0±3.6%). Lysozyme cleaning efficacy was demonstrated by the test solution, which in the absence of surfactant is believed to function through an ion-exchange mechanism.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. An ophthalmic composition comprising hydrogen peroxide and a boron compound, said composition having a pH of between 6.5 and 9.0.

2. A composition according to claim 1 having a hydrogen peroxide concentration of 0.1 to 3.5 w/v % and a boron compound selected from the group consisting of sodium borate, boric acid, and combinations thereof.

3. A composition according to claim 2, wherein the total boron concentration of said composition is between 0.05M and 0.15M.

4. A composition according to claim 1, said composition having a pH of 7.0 to 7.9.

5. A composition according to claim 1, said composition having a pH of 7.0 to 7.5.

6. A composition according to claim 1, said composition haying an osmolality of 210 to 320 mOsm/kg.

7. A composition according to claim 1, said composition being substantially free of surfactants.

8. A composition according to claim 1, said composition further comprising an indicator compound.

9. A composition according to claim 1, further comprising monobasic sodium phosphate, dibasic sodium phosphate, and sodium chloride.

10. An improved ophthalmic composition comprising hydrogen peroxide, said composition further comprising a concentration of boron sufficient to reduce or prevent microbial growth in the composition following neutralization of the hydrogen peroxide.

11. A composition according to claim 10, said composition having a pH of 7.0 to 7.5.

12. A composition according to claim 10, said composition having a total boron concentration of 0.10M to 0.15M.

13. A composition according to claim 12, said composition being substantially free of surfactants.

14. A composition according to claim 12, said composition further comprising an indicator compound.

15. In a method for disinfecting contact lenses comprising immersing a contact lens in an ophthalmic composition comprising hydrogen peroxide, the improvement consisting of immersing a contact lens in an ophthalmic composition comprising hydrogen peroxide and a boron compound, said boron compound present in a concentration sufficient to reduce or prevent microbial growth in the composition.

16. A method according to claim 15, wherein said composition has a total boron concentration of 0.05M to 0.15M.

17. A method according to claim 16, wherein said composition has a total boron concentration of 0.10M to 0.15M.

18. A method according to claim 17, wherein said composition is substantially free of surfactants.

19. A method according to claim 17, wherein said composition further comprises an indicator compound.

20. An ophthalmic composition consisting essentially of:
a) 3.0 w/v % hydrogen peroxide;
b) 0.33 w/v % sodium borate;
c) 0.41 w/v % boric acid;
d) 0.136 w/v % monobasic sodium phosphate;
e) 0.062 w/v % dibasic sodium phosphate;
f) 0.12 w/v % DEQUEST® 2060S;
g) 0.47 w/v % sodium chloride;
h) a pH-adjusting agent in an amount sufficient to cause the composition to have a pH of 7.0; and
i) purified water.
